(19) United States
(12) Patent Application Publication
(10) Pub. No.: US 2008/0279956 A1
(43) Pub. Date: Nov. 13, 2008

(54) Method for collecting a live placenta cord stem cell

(76) Inventor: Tung-Ho Lin, Tainan City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/797,929

(22) Filed: May 9, 2007

(51) Int. Cl. A61K 35/14

(52) U.S. Cl. 424/529

(57) ABSTRACT

The present invention discloses a method for collecting a live placenta cord stem cell, in which the live placenta cord stem cells are required to be healthy and plenty of endocrine. The cord is first picked with a proper length, then dipped in the sodium citrate solution of a specific concentration as an anticoagulant and then preserved in a refrigerator to maintain natural activity thereof. The collected stem cells can be implanted into human bodies without synthetic chemicals, side effects and rejection, and therefore are suitable for treating many diseases.

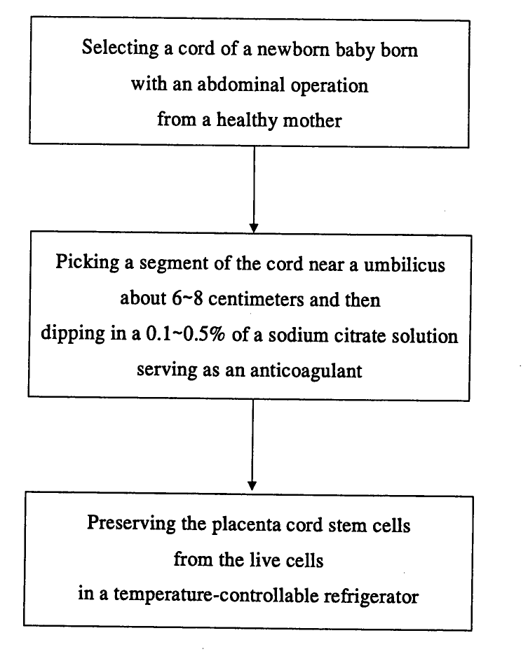

### BACKGROUND OF THE INVENTION

1. Field of the Invention

The present invention relates to a method for collecting a stem cell, and particularly to a method for collecting a live placenta cord stem cell without implanting the stem cell into a human body. In the present invention, the placenta cord stem cells collected from the live cells can effectively maintain main structures of cell proliferation factors without destroying activity of the placenta. Therefore, the live placenta cord stem cells of the present invention are suitable for treating many diseases.

2. Related Prior Arts

As an ancient medicine, the placenta has been recorded as “Bao-I” in “Ben Tsao Hse I” at Tang Dynasty and as “Tse-Ho-Cho” in “Ben Tsao Kang Mu” at Ming Dynasty. The placenta can be used to implement blood and spirit, detoxify and calm nerves. People usually take it for sweat, premature ejaculation, impotence, facial, cough after cooking, stewing, packaging in dumplings or drying and grinding to powders.

Commercially, the stem cells of the placenta can be extracted by means of: (1) adding water and decomposition, (2) enzyme decomposition or (3) molecular division. The stem cells are then used to produce placental globulins, placental tissue fluid, placenta tissue serum, etc., which can be used for promoting immunity or anti-allergy and can be in the forms of cosmetic lotion, oral tablet, injection, etc.

However, though the above placental production can be effective on treating many diseases, maintaining health, anti-aging and preventing or fighting cancer, their therapeutic effects are dependent on chemical synthesis process and results. In addition, such extraction manners could not maintain stereo structures of cell proliferation factors and reduce placental activity, so that these traditional product can not be widely applied.

### SUMMARY OF THE INVENTION

In the present invention, the method for collecting a live placenta cord stem cell is to collect stem cells from a cord, but not to implant stem cells into a human body by operation. Basically, the live placenta cord stem cells are required to be healthy and plenty of endocrine. The cord is first picked with a proper length, then dipped in the sodium citrate solution of a specific concentration as an anticoagulant and then preserved in a refrigerator to maintain natural activity thereof. The collected stem cells can be implanted into human bodies without synthetic chemicals, side effects and rejection, and therefore are suitable for treating many diseases.

### BRIEF DESCRIPTION OF THE DRAWINGS

The FIGURE shows the process of the present invention.

### DETAILED DESCRIPTION OF THE PREFERRED EMBODIMENTS

First, please refer to the FIGURE which shows the process for collecting the live placenta cord stem cells of the present invention:

(1) selecting a pregnant woman who is healthy without suffering from diabetes, diseases about liver or kidney, hepatitis, pathogen, and performing an abdominal operation on the woman to bear a neonatal baby whose cord will be plenty of endocrine;

(2) picking a 6˜8 cm segment of the baby's cord near his umbilicus and then dipping the picked cord in a sodium citrate solution having a concentration 0.1˜0.5 wt. % and serving as an anticoagulant; and

(3) storing the cord with the solution in a temperature-controllable refrigerator for preserving live placenta cord stem cells of the cord, wherein the live placenta cord stem cells can be preserved for 2˜10 days at 5˜−20° C., or forever below −270° C. in a liquefied nitrogen refrigerator. The cord can be preserved for 30 years in the current equipment for storing cord blood, and has to be moved to the refrigerator as soon as possible after picked, for example, within 6˜8 hours, so that activity of the live cells can be maintained.

In the above steps (2) and (3), the picked cord is preferably near the baby's umbilicus and 6˜8 cm long, then dipped in a sodium citrate solution of 0.2 wt. % as an anticoagulant solution, and then stored in the refrigerator at −5° C. for 3 days.

Further, in the above steps (2) and (3), the picked cord is preferably near the baby's umbilicus and about 7 cm long, then dipped in a sodium citrate solution of 0.3 wt. % as an anticoagulant solution, and then stored in the refrigerator at −˜−15° C. for 7 days.

Further, in the above steps (2) and (3), the picked cord is preferably near the baby's umbilicus and about 8 cm long, then dipped in a sodium citrate solution of 0.5 wt. % as an anticoagulant solution, and then stored in the refrigerator at −15˜−20° C. for 10 days.

The method for collecting the live placenta cord stem cells of the present invention exhibits advantages as the live placenta cord stem cells includes substances as follows:
1. protein, fat, carbohydrate and various enzymes (for example, β-N-acetyl-D-procainamase, β-inulin aldehydase, alcohol dehydrogenase, Polyol dehydrogenase, sorbic acid dehydrogenase, lactate dehydrogenase, malate dehydrogenase, isocitrate dehydrogenase, phosphoglycolate dehydrogenase, glucose phosphate dehydrogenase, hydroxysteroid dehydrogenase, xanthine oxidase, succinate dehydrogenase, glutamate dehydrogenase, monoamine oxidase, diamine oxidase, tolidine nucleotide transferase, NADPH cytochrome C reductase, NADH diaphorase, NADPH diaphorase, cytochrome oxidase, catalase, steroid hydroxylase, imidazolium methyl transferase, estrogen methyl transferase, Omithine transferase, transketolase, transaldolase, choline Acetyltransferase, acetyl glycerophosphate choline, acetyl transferase, UDP, gluconoic transferase, methionine adenosyl transferase, thiaminase, aspartate aminotransferase (oxaloacetate glutaminase), alanine aminotranferease (glutate pyruv aminotransferase), tyrosine aminotransferase, hexokinase, fructokinase, phosphofructokinase, glycerol kinase, NAD kinase, creatine kinase, AMP kinase, phosphofructose kinase, ribonulease, arylsulfotransferase, carboxylesterase, esterase, lipoprteinlipase, phospholipase, acetylesterase, TAP, phosphatase, glucophosphatase, phosphodiesterase, DNase, allylsulphatase, sterolsulphatase, lysozyme, glucoside hydrolase, LAP, cystineaminopeptidase, hydroxypeptidase, glycyglycindipeptidase, glycyleucinedipeptidase, glycyprolinedipeptidase, rennin, peptidase, glutamase, ureazyme, arqinase, GUA, adenosine deaminase, inorganic pyrophosphatase, adenosine triphosphatase, acid phosphatase, nucleotide pyrophosphatase, malon-COA decarboxylase, serotonate decarboxylase, phytosterol 20-22 lyase, steroid 17-20 lyase, fumarase, aconitase, histidine ammonia lyase, tyrosine deiodinase, ribulose pentophosphate epimerase, ribulose pentophosphate isomerase, malon-COA synthase, steroidaromatase, glucanase, fatty acid, synthase, glycerol triacylglycerols synthase, lipid synthase, protein synthase, RNA synthase, DNA synthase), mineral, vitamin, and the identified components include nucleic acid such as adenine, guanine, uracil, urinary nucleotide, xanthine, hypoxanthine, guanosine, adenosine, etc.;
2. polypeptide;
3. glycosaminglycan, hyaluronate chondroitin, etc.;
4. amino acid: arquinine, aspartate, glutamate, proline, alanine, methionine, isoleucine, etc.

The cord stem cells serve as channel for delivering nucleic acid and amino acid. Effects of the nucleic acid in human bodies include: (1) assisting the taken nutrition, particularly protein, to be highly digested and absorbed for proliferation of tissues; (2) promoting growth of the hematopoietic organs; (3) promoting regeneration of tissues of the organs such as liver; (4) promoting babies' development; (5) enhancing lactation; (6) adjusting self-conscious nerves; (7) enhancing and normalizing hormones secretion; (8) enhancing recovery of wounds; (9) enhancing formation of glycogen; (10) enhancing blood circulation.

According to investigations and identification, relationships between the cord growth factors and the cytokinins are as follows:
HGF proliferation factor of liver cells, enhancing proliferation of liver cells;
NGF proliferation factor of nerve cells, enhancing proliferation of nerve cells;
FGF proliferation factor of fibrolasts, enhancing proliferation of fibrolasts, nerve cytes, vessel endothelial cells;
EGF proliferation factor of epithelia, enhancing proliferation of epithelia of skins, lung, cornea, trachea;
CSF stimulating factor for forming colonies, enhancing proliferation of stem cells such as granular cells, macrophages, etc.;
IGF growth factor of insulin, enhancing growth of chondrocytes, smooth muscle cells;
TGF proliferation factor of form transformation, transforming reversible non-form cells into form cells;
IL₁ leukocyte bactericide₁, enhancing proliferation of enhancing immunocytes (T, B, NK) thymocytes;
IL₂ leukocyte bactericide₂, enhancing proliferation of T cells, enhancing generation of lymph cytokines;
IL₃ leukocyte bactericide₃, enhancing proliferation of hematopoietic cells, obese cells;
IL₄ leukocyte bactericide₄, enhancing proliferation of B cells, and differentiation of antibody production cells.

So far, eighteen types of leukocyte bactericides are identified.

When implanting the placenta cord stem cells collected by the method of the present invention into human bodies, matching of blood groups is not necessary as blood has been removed during collecting the stem cells. Such method will perform advantages as follows:
1. repairing damaged organs and cells;
2. initiating the immune system again to promote immunity;
3. enhancing endocrine of all organs.

The stem cells can used to treat many diseases, for example, asthma, allergic rhinitis, female climacteric syndrome, male sexual dysfunction, promoting immunity, enhancing resistance, preventing cancer, hepatitis A, B and C, liver cirrhosis, chronic nephritis, gastric syndrome, psychiatric gastric ulcer, neurasthenia, facial, promoting voice sound, anti-aging, rejuvenation, etc.

Statistic data for clinic patients applying the stem cells of the present invention are as follows:
(1) asthma: total thirty seven patients, wherein four are 15˜20 years old (two men and two women); three are 30˜40 years old (two men and one woman); sixteen are 40˜50 years old (nine men and seven women); seven are 50˜60 years old (three men and four women); four are 60˜70 years old (two men and two women); three are 70˜80 years old (three women); thirty one are cured, six are improved, one is ineffective, and cure rate is 83.7%.
(2) allergic rhinitis: total fifteen patient, wherein five are 20˜30 years old (three men and two women); seven are 30˜50 years old (four men and three women); three are 50˜70 years old (one man and two women); twelve are cured, two are improved, one is ineffective, and cure rate is 86%.
(3) neurasthenia: total twenty eight patients at the age of 25˜50 years old (twenty men and eight women), twenty four are cured, two are improved, two are ineffective, and cure rate is 85.7%.
(4) climacteric syndrome: total fifteen women are 35˜50 years old; thirteen are cured, one is improved, one is ineffective, and cure rate is 86.6%.

(5) gastric syndrome: total five patients at the age of 32˜39 years old (two men and three women); four are cured, one is improved, and cure rate is 80%.
(6) hypertension cardiac: total eight patients at the age of 45˜73 years old (four men and four women); five are cured, three are improved, and cure rate is 62.5%.
(7) hepatitis: total seven patients at the age of 31˜45 years old (five men and two women); six are cured, one is improved, and cure rate is 85.7%.
(8) anti-aging: total fifteen patients at the age of 20˜62 years old (eight men and seven women), effect rate is 100%.

As disclosed in the preferred embodiments, the present invention is effective as desired and never mentioned in the prior arts or existent product. It should be notice that the preferred embodiments are provided to explain the invention and the scope of the invention will be referred to claims.

What is claimed is:

1. A method for collecting a live placenta cord stem cell, comprising steps of:
(1) selecting a pregnant woman and performing an abdominal operation on the woman to bear a baby;
(2) picking a 6˜8 cm segment of the baby's cord near his umbilicus and then dipping the picked cord in a sodium citrate solution having a concentration 0.1˜0.5 wt. % and serving as an anticoagulant; and
(3) storing the cord with the solution in a temperature-controllable refrigerator for preserving live placenta cord stem cells of the cord.

2. The method of claim 1, wherein the refrigerator is controlled at a temperature ranging from 5° C. to −20° C. in the step (3) and the live placenta cord stem cells are preserved for 3˜10 days.
